SEQUENCE LISTING (73 sequences)

SEQ ID NO: 1 - DNA, 18 bp, synthetic construct
  source 1..18: mol_type = other DNA
atttaggtgacactatag

SEQ ID NO: 2 - protein, 874 aa, Salmonella phage SP6
MQDLHAIQLQLEEEMFNGGIRRFEADQQRQIAAGSESDTAWNRRLLSELIAPMAEGIQAYKEEYEGKKGRAPRALAFLQCVENEVAAYITMKVVMDMLNTDATLQAIAMSVAERIEDQVRFSKLEGHAAKYFEKVKKSLKASRTKSYRHAHNVAVVAEKSVAEKDADFDRWEAWPKETQLQIGTTLLEILEGSVFYNGEPVFMRAMRTYGGKTIYYLQTSESVGQWISAFKEHVAQLSPAYAPCVIPPRPWRTPFNGGFHTEKVASRIRLVKGNREHVRKLTQKQMPKVYKAINALQNTQWQINKDVLAVIEEVIRLDLGYGVPSFKPLIDKENKPANPVPVEFQHLRGRELKEMLSPEQWQQFINWKGECARLYTAETKRGSKSAAVVRMVGQARKYSAFESIYFVYAMDSRSRVYVQSSTLSPQSNDLGKALLRFTEGRPVNGVEALKWFCINGANLWGWDKKTFDVRVSNVLDEEFQDMCRDIAADPLTFTQWAKADAPYEFLAWCFEYAQYLDLVDEGRADEFRTHLPVHQDGSCSGIQHYSAMLRDEVGAKAVNLKPSDAPQDIYGAVAQVVIKKNALYMDADDATTFTSGSVTLSGTELRAMASAWDSIGITRSLTKKPVMTLPYGSTRLTCRESVIDYIVDLEEKEAQKAVAEGRTANKVHPFEDDRQDYLTPGAAYNYMTALIWPSISEVVKAPIVAMKMIRQLARFAAKRNEGLMYTLPTGFILEQKIMATEMLRVRTCLMGDIKMSLQVETDIVDEAAMMGAAAPNFVHGHDASHLILTVCELVDKGVTSIAVIHDSFGTHADNTLTLRVALKGQMVAMYIDGNALQKLLEEHEERWMVDTGIEVPEQGEFDLNEIMDSEYVFA

SEQ ID NO: 3 - DNA, 18 bp, synthetic construct
  source 1..18: mol_type = other DNA
taatacgactcactatag

SEQ ID NO: 4 - DNA, 32 bp, synthetic construct
  source 1..32: mol_type = other DNA
aagctcttcatccggagaattctaatacgact

SEQ ID NO: 5 - DNA, 32 bp, synthetic construct
  source 1..32: mol_type = other DNA
ttgctcttcttcgaccttttctcctcttttct

SEQ ID NO: 6 - DNA, 37 bp, synthetic construct
  source 1..37: mol_type = other DNA
ttgctcttcttcgcatttttctcctcttttctctctc

SEQ ID NO: 7 - DNA, 32 bp, synthetic construct
  source 1..32: mol_type = other DNA
aagctcttcatccaggtatgcgcgaagcggtt

SEQ ID NO: 8 - DNA, 32 bp, synthetic construct
  source 1..32: mol_type = other DNA
ttgctcttcttcgaagcttacttgccgaccac

SEQ ID NO: 9 - DNA, 40 bp, synthetic construct
  source 1..40: mol_type = other DNA
tctgcgctctgctgaagccagttaccttcggaaaaagagt

SEQ ID NO: 10 - DNA, 40 bp, synthetic construct
  source 1..40: mol_type = other DNA
gtactcacttcagcaataaccgcttagcgcatacctcgag

SEQ ID NO: 11 - DNA, 40 bp, synthetic construct
  source 1..40: mol_type = other DNA
aggtcaatatcgctatgtggctacaagcccccatcgaccg

SEQ ID NO: 12 - DNA, 43 bp, synthetic construct
  source 1..43: mol_type = other DNA
caataacaccgaccacttcagattactgggtactcacttcagc

SEQ ID NO: 13 - DNA, 40 bp, synthetic construct
  source 1..40: mol_type = other DNA
catcgaccgcggaaccatactaatgtaccgctagtaaagt

SEQ ID NO: 14 - DNA, 50 bp, synthetic construct
  source 1..50: mol_type = other DNA
cttcagcaataaccgcttcgcggatacctcgagaccagaagaccgaattc

SEQ ID NO: 15 - DNA, 50 bp, synthetic construct
  source 1..50: mol_type = other DNA
cttcagcaataaccgcttcgcgcagacctcgagaccagaagaccgaattc

SEQ ID NO: 16 - DNA, 50 bp, synthetic construct
  source 1..50: mol_type = other DNA
cttcagcaataaccgcttcgcgcgtacctcgagaccagaagaccgaattc

SEQ ID NO: 17 - DNA, 40 bp, synthetic construct
  source 1..40: mol_type = other DNA
caccgaccacttcagataactaggtactcacttcagcaat

SEQ ID NO: 18 - DNA, 40 bp, synthetic construct
  source 1..40: mol_type = other DNA
tcaagcccccatcgaccgcttaaccatacaaatgtaccgc

SEQ ID NO: 19 - DNA, 40 bp, synthetic construct
  source 1..40: mol_type = other DNA
cgcgtttagccggataccgctaaggaatgatatcatcatg

SEQ ID NO: 20 - DNA, 40 bp, synthetic construct
  source 1..40: mol_type = other DNA
actctttttccgaaggtaactggcttcagcagagcgcaga

SEQ ID NO: 21 - DNA, 40 bp, synthetic construct
  source 1..40: mol_type = other DNA
ctcgaggtatgcgctaagcggttattgctgaagtgagtac

SEQ ID NO: 22 - DNA, 40 bp, synthetic construct
  source 1..40: mol_type = other DNA
cggtcgatgggggcttgtagccacatagcgatattgacct

SEQ ID NO: 23 - DNA, 43 bp, synthetic construct
  source 1..43: mol_type = other DNA
gctgaagtgagtacccagtaatctgaagtggtcggtgttattg

SEQ ID NO: 24 - DNA, 40 bp, synthetic construct
  source 1..40: mol_type = other DNA
actttactagcggtacattagtatggttccgcggtcgatg

SEQ ID NO: 25 - DNA, 50 bp, synthetic construct
  source 1..50: mol_type = other DNA
gaattcggtcttctggtctcgaggtatccgcgaagcggttattgctgaag

SEQ ID NO: 26 - DNA, 50 bp, synthetic construct
  source 1..50: mol_type = other DNA
gaattcggtcttctggtctcgaggtctgcgcgaagcggttattgctgaag

SEQ ID NO: 27 - DNA, 50 bp, synthetic construct
  source 1..50: mol_type = other DNA
gaattcggtcttctggtctcgaggtacgcgcgaagcggttattgctgaag

SEQ ID NO: 28 - DNA, 40 bp, synthetic construct
  source 1..40: mol_type = other DNA
attgctgaagtgagtacctagttatctgaagtggtcggtg

SEQ ID NO: 29 - DNA, 40 bp, synthetic construct
  source 1..40: mol_type = other DNA
gcggtacatttgtatggttaagcggtcgatgggggcttga

SEQ ID NO: 30 - DNA, 40 bp, synthetic construct
  source 1..40: mol_type = other DNA
catgatgatatcattccttagcggtatccggctaaacgcg

SEQ ID NO: 31 - DNA, 25 bp, synthetic construct
  source 1..25: mol_type = other DNA
aagctcttcatccggagaattcatt

SEQ ID NO: 32 - DNA, 25 bp, synthetic construct
  source 1..25: mol_type = other DNA
ttgctcttcttcgaccttttctcct

SEQ ID NO: 33 - DNA, 34 bp, synthetic construct
  source 1..34: mol_type = other DNA
aagctcttcatccaatgaccaacctttccgacat

SEQ ID NO: 34 - DNA, 38 bp, synthetic construct
  source 1..38: mol_type = other DNA
ttgctcttcttcgaagcttacagcatcttgatcttgtt

SEQ ID NO: 35 - DNA, 51 bp, synthetic construct
  source 1..51: mol_type = other DNA
aagctcttcatccaatgagtgaagttgaattcagccatgaatattggatgc

SEQ ID NO: 36 - DNA, 38 bp, synthetic construct
  source 1..38: mol_type = other DNA
ttgctcttcttcgacctaaactttcgggtgtggcggac

SEQ ID NO: 37 - DNA, 43 bp, synthetic construct
  source 1..43: mol_type = other DNA
aagctcttcatccggagaagaaaccaattgtccatattgcatc

SEQ ID NO: 38 - DNA, 29 bp, synthetic construct
  source 1..29: mol_type = other DNA
aagctcttcatgccagtaaaagacgcagt

SEQ ID NO: 39 - DNA, 30 bp, synthetic construct
  source 1..30: mol_type = other DNA
aagctcttcagcacttctcgctaaccaaac

SEQ ID NO: 40 - DNA, 41 bp, synthetic construct
  source 1..41: mol_type = other DNA
ttgctcttcttcgcatttatggagaaacagtagagagttgc

SEQ ID NO: 41 - DNA, 37 bp, synthetic construct
  source 1..37: mol_type = other DNA
aagctcttcatccggagcaaatggacgaagcagggat

SEQ ID NO: 42 - DNA, 29 bp, synthetic construct
  source 1..29: mol_type = other DNA
aagctcttcatgccagtaaaagacgcagt

SEQ ID NO: 43 - DNA, 30 bp, synthetic construct; identical to SEQ ID NO: 39
  source 1..30: mol_type = other DNA

SEQ ID NO: 44 - DNA, 36 bp, synthetic construct
  source 1..36: mol_type = other DNA
ttgctcttcttcgcatttgccatggtgaattcctcc

SEQ ID NO: 45 - DNA, 30 bp, synthetic construct
  source 1..30: mol_type = other DNA
aagctcttcactccggcttcatcggtagtt

SEQ ID NO: 46 - DNA, 30 bp, synthetic construct
  source 1..30: mol_type = other DNA
aagctcttcagtccggcttcatcggtagtt

SEQ ID NO: 47 - DNA, 31 bp, synthetic construct
  source 1..31: mol_type = other DNA
aagctcttcagaggacatcgatatgaacccc

SEQ ID NO: 48 - DNA, 31 bp, synthetic construct
  source 1..31: mol_type = other DNA
aagctcttcagtatcttccaggacttgttgc

SEQ ID NO: 49 - DNA, 30 bp, synthetic construct
  source 1..30: mol_type = other DNA
aagctcttcataccattcagcctgcgatcg

SEQ ID NO: 50 - DNA, 32 bp, synthetic construct
  source 1..32: mol_type = other DNA
ttgctcttcttcgaagcttaggcgaatgcgaa

SEQ ID NO: 51 - DNA, 37 bp, synthetic construct
  source 1..37: mol_type = other DNA
aagctcttcatccggagctggctaaatacggaaggaa

SEQ ID NO: 52 - DNA, 42 bp, synthetic construct
  source 1..42: mol_type = other DNA
ttgctcttcttcgcattctagtatttctcctcaatttcatta

SEQ ID NO: 53 - DNA, 41 bp, synthetic construct
  source 1..41: mol_type = other DNA
aagctcttcatccaggtaacacgattaacatcgctaagaac

SEQ ID NO: 54 - DNA, 35 bp, synthetic construct
  source 1..35: mol_type = other DNA
ttgctcttcttcgaagcttacgcgaacgcgaagtc

SEQ ID NO: 55 - DNA, 41 bp, synthetic construct
  source 1..41: mol_type = other DNA
aagctcttcatccaatgaacacgattaacatcgctaagaac

SEQ ID NO: 56 - DNA, 37 bp, synthetic construct
  source 1..37: mol_type = other DNA
gcatggtctcaggagcctattctggtggaactggatg

SEQ ID NO: 57 - DNA, 36 bp, synthetic construct
  source 1..36: mol_type = other DNA
gcatggtctcaggaggaaggcgataccctggtaaac

SEQ ID NO: 58 - DNA, 36 bp, synthetic construct
  source 1..36: mol_type = other DNA
gcatggtctcaggagcgcaaagcgttctgtctaaag

SEQ ID NO: 59 - DNA, 35 bp, synthetic construct
  source 1..35: mol_type = other DNA
gcatggtctcaggaggctgaccctgaagttcatct

SEQ ID NO: 60 - DNA, 33 bp, synthetic construct
  source 1..33: mol_type = other DNA
gcatgctcttcaatgcaaggaggacggcaacat

SEQ ID NO: 61 - DNA, 37 bp, synthetic construct
  source 1..37: mol_type = other DNA
gcatgctcttcaatggagttcgaaatcgaaggtgaag

SEQ ID NO: 62 - DNA, 37 bp, synthetic construct
  source 1..37: mol_type = other DNA
gcatgctcttcaatgcgagtgaaacacaggaagatca

SEQ ID NO: 63 - DNA, 37 bp, synthetic construct
  source 1..37: mol_type = other DNA
gcatgctcttcaatgctgcgccaattgatgacaatac

SEQ ID NO: 64 - DNA, 37 bp, synthetic construct
  source 1..37: mol_type = other DNA
gcatggtctcaagcgtgcttcatatggtccggataac

SEQ ID NO: 65 - DNA, 35 bp, synthetic construct
  source 1..35: mol_type = other DNA
gcatggtctcaagcgaacaggaccatcaccgattg

SEQ ID NO: 66 - DNA, 36 bp, synthetic construct
  source 1..36: mol_type = other DNA
gcatggtctcaagcgtgagccagtgtgactctagta

SEQ ID NO: 67 - DNA, 34 bp, synthetic construct
  source 1..34: mol_type = other DNA
gcatggtctcaagcggtcttgtagttgccgtcgt

SEQ ID NO: 68 - DNA, 35 bp, synthetic construct
  source 1..35: mol_type = other DNA
gcatgctcttcaaccggactggtagctcaggtagt

SEQ ID NO: 69 - DNA, 36 bp, synthetic construct
  source 1..36: mol_type = other DNA
gcatgctcttcaacccgttcccatttgaaaccttcc

SEQ ID NO: 70 - DNA, 35 bp, synthetic construct
  source 1..35: mol_type = other DNA
gcatgctcttcaaccaccgttgcccttctcaatac

SEQ ID NO: 71 - DNA, 37 bp, synthetic construct
  source 1..37: mol_type = other DNA
gcatgctcttcaaccctgaaaccacagccacattaac

SEQ ID NO: 72 - DNA, 28 bp, synthetic construct
  source 1..28: mol_type = other DNA
annnnnnnctcgggcaaatcgctgaata

SEQ ID NO: 73 - DNA, 33 bp, synthetic construct
  source 1..33: mol_type = other DNA
tnnnnnnncttgtgcaatgtaacatcagagatt

(19) United States
(12) Patent Application Publication
(10) Pub. No.: US 2026/0308569 A1
(43) Pub. Date: Oct. 8, 2026

(54) System, Kit, and Method for Continuous Directed Evolution

(71) Applicant: The Board of Trustees of the University of Illinois, Urbana, IL (US)

(72) Inventors: Steven Burgess, Urbana, IL (US); Ignacio Sparrow Munoz, Urbana, IL (US)

(21) Appl. No.: 19/637,099

(22) Filed: Apr. 2, 2026

Related U.S. Application Data
(60) Provisional application No. 63/784,396, filed on Apr. 7, 2025.

(51) Int. Cl. C12N 15/10; C12N 9/12; C12N 9/78

(52) U.S. Cl. CPC C12N 15/1058; C12N 9/1247; C12N 9/78; C12Y 207/07006; C12Y 305/04001; C12Y 305/04002

(57) ABSTRACT

A system, kit, and method for continuous directed evolution are provided, which include the use of a first expression construct comprising a SP6 promoter operably linked to a polynucleotide encoding a first protein of interest; and a second expression construct comprising at least one polynucleotide encoding a SP6 RNA polymerase-deaminase fusion protein.

### REFERENCE TO RELATED APPLICATIONS

This application claims the benefit of priority from U.S. Provisional Application Ser. No. 63/784,396, filed Apr. 7, 2025, the content of which is incorporated herein by reference in its entirety.

### FEDERALLY SPONSORED RESEARCH AND DEVELOPMENT

This invention was made with government support under DE-SC0018420 awarded by the Department of Energy. The government has certain rights in the invention.

### REFERENCE TO AN ELECTRONIC SEQUENCE LISTING

The contents of the electronic sequence listing (name: IL0049US_ST26.xml; size: 67,266 bytes; and date of creation: Mar. 26, 2026) is herein incorporated by reference in its entirety.

### BACKGROUND OF THE INVENTION

Protein engineering, including the production of improved enzymes, has the potential to assist in addressing societal challenges from development of green chemistry to the conversion of lignocellulosic biomass into useful products. Due to the complexity of protein-structure function relationships engineering methods often rely on experimental diversification followed by screening, known as directed evolution. Classical approaches involve iterative cycles of in vitro genetic diversification and selection for altered function. However, this can be time consuming and labor intensive. Continuous directed evolution systems have been developed to overcome these limitations and involve in vivo mutation and selection of target sequences, with protein function coupled to a measurable phenotype, such as growth or fluorescence (Wang et al. (2021) Chem. Rev. 121(20):12384-444; You et al. (1996) Protein Eng. Des. Sel. 9(1):77-83; Garcia-Garcia et al. (2022) Plant Physiol. 188(2):971-83; Rix et al. (2020) Nat. Commun. 11(1):5644; Ravikumar et al. (2018) Cell 175(7):1946-1957.e13; Kuchner & Arnold (1997) Trends Biotechnol. 15(12):523-30; Packer & Liu (2015) Nat. Rev. Genet. 16(7):379-94). Each system has advantages and limitations, but ease of use, and adaptability to a variety of host system are likely to be important for adoption. One approach has involve mutating a gene of interest using a deaminase-fused to T7 RNA polymerase (Moore et al. (2018) J. Am. Chem. Soc. 140(37):11560-4; Mengiste et al. (2023) Nucleic Acids Res. 51(6):e31; Park & Kim (2021) Nucleic Acids Res. 49(6):e32; Seo et al. (2023) Nucleic Acids Res. 51(10):e59; Cravens et al. (2021) Nat. Commun. 12(1):1579; WO 2019/183049). All the machinery is contained within a plasmid, requiring only the cloning the gene of interest into target vector. This approach can be used for any organism in which T7 can be used as an expression system, such as cyanobacteria, yeast or mammalian and plant cells.

However, current T7-based systems are tailored to specific organisms, and are based on conventional restriction digest cloning, which presents a barrier to implementation in new species. Accordingly, improved systems for continuous directed evolution are needed.

### SUMMARY OF THE INVENTION

The present invention provides a system for continuous directed evolution, comprising (a) a first expression construct comprising a SP6 promoter operably linked to a polynucleotide encoding a first protein of interest; and (a) a second expression construct comprising at least one polynucleotide encoding a SP6 RNA polymerase-deaminase fusion protein.

Also provided is a kit comprising (a) a polynucleotide encoding a SP6 RNA polymerase, (b) a polynucleotide encoding a cytosine deaminase and/or, a polynucleotide encoding a adenine deaminase, and (c) a SP6 promoter.

Further provided is a method of continuous directed evolution of a protein of interest, comprising (a) introducing into a host cell a first expression construct comprising a SP6 promoter operably linked to a polynucleotide encoding the protein of interest; (b) introducing into the host cell a second expression construct comprising at least one polynucleotide encoding a SP6 RNA polymerase-deaminase fusion protein; and (c) culturing the host cell under conditions suitable for continuous directed evolution of the protein of interest.

### BRIEF DESCRIPTION OF THE DRAWINGS

FIGS. 1A-1C show the characterization of Golden Gate assembled, SP6-driven continuous directed evolution system in E. coli strain 5a. FIG. 1A, Schematic of constructs used. p15 indicates p15 backbone (low copy) and pCA indicates pCAMBIA backbone (high copy). Vertical hatches in constructs indicate Golden Gate assembly. FIG. 1B, Log suppressor frequency of the indicated constructs before and after 2 passages (n=4 biological replicates). FIG. 1C, mutation counts of the indicated constructs at P2. Significance calculated through Kruskal-Wallis test followed by a post-hoc Dunn's test (n=8 biological replicates) with the Bonferroni correction. ** and *** denote p<0.01 and p<0.001 in relation to the empty vector control, respectively. n.s.=non-significant.

### DETAILED DESCRIPTION OF THE INVENTION

A system, kit, and method for continuous directed evolution have now been developed that uses the viral RNA polymerase from Salmonella enterica phage SP6. The system, kit, and method may be used in conjunction with a standardized modular cloning assembly library, which uses the syntax of the Plant Golden Gate modular cloning toolkit (Engler et al. (2014) ACS Synth. Biol. 3(11):839-843) thereby enabling users to assemble custom vectors for directed evolution by varying mutators, promoters, and proteins of interest. The system may be used in in bacterial and photosynthetic host cells. In addition, the system, kit, and method may further include conventional T7 RNA polymerase-based vectors to allow for greater flexibility when chaperones or other heterologous proteins may need to be expressed to enable evolution schemes.

Accordingly, in one aspect, a system for continuous directed evolution is provided, which comprises a first expression construct comprising a SP6 promoter operably linked to a polynucleotide encoding a first protein of interest; and (a) a second expression construct comprising at least one polynucleotide encoding a SP6 RNA polymerase-deaminase fusion protein. As used herein, “continuous directed evolution” refers to an evolution procedure, in which a population of nucleic acids is subjected to multiple rounds of (a) replication, (b) mutation (or modification of the primary sequence of nucleotides of the nucleic acids in the population), and (c) selection to produce a desired evolved product, for example, a novel nucleic acid encoding a novel protein with a desired activity, wherein the multiple rounds of replication, mutation, and selection can be performed without investigator interaction, and wherein the processes (a)-(c) can be carried out simultaneously. Typically, the evolution procedure is carried out in vivo or in vitro, for example, using cells in culture as host cells. In some aspects, a continuous directed evolution process provided herein relies on a system in which a polynucleotide encoding a protein of interest is provided in an expression construct or vector that undergoes a life-cycle including replication in a host cell and transfer to another host cell, wherein a critical component of the life-cycle is deactivated and reactivation of the component is dependent upon a desired variation in an amino acid sequence of a protein encoded by the gene of interest.

An “expression construct” refers to a vector such as a recombinant vector that is suitable for expression of a polynucleotide encoding a protein of interest in an organism. “Expression” refers to the production of a functional product. In some aspects, expression of a polynucleotide sequence may refer to the transcription of a polynucleotide sequence (e.g., transcription to produce mRNA or functional RNA) and the translation of an RNA into a precursor or mature protein. In some aspects, an “expression construct” herein may be a circular plasmid or vector.

If not specified, it is to be understood that an expression construct herein includes regulatory sequences or elements necessary for expression of a polynucleotide encoding a protein of interest. A “regulatory sequence” and “regulatory element” refers to a nucleotide sequence that is located upstream (5′ non-coding sequence), in the middle, or downstream (3′ non-coding sequence) of a coding sequence and affects the transcription, RNA processing or stability, and/or translation of the relevant coding sequence.

Regulatory sequences may include, but are not limited to, promoters, enhancers, translation leaders, introns, and polyadenylation recognition sequences.

The term “promoter” refers to a polynucleotide with a sequence recognized by the cellular transcription machinery and able to initiate transcription of a downstream nucleic acid sequence. A promoter may be constitutively active, meaning that the promoter is always active in a given cellular context, or conditionally active, meaning that the promoter is only active under specific conditions. For example, a conditional promoter may only be active in the presence of a specific protein that connects a protein associated with a regulatory element in the promoter to the basic transcriptional machinery, or only in the absence of an inhibitory molecule. A subclass of conditionally active promoters are inducible promoters that require the presence of a stimulus for activity. Examples of inducible promoters that facilitate conditional gene expression are known in the art. For example, inducible promoters may be chemically regulated (e.g., alcohol-regulated, tetracycline-regulated, steroid-regulated, or metal-regulated). Other inducible promoters may be physically regulated (e.g., temperature-regulated or light-regulated). In some aspects, an inducible promoter may include, but is not limited to, an arabinose-inducible promoter, rhamnose inducible promoter, Tet-on promoter, or tamoxifen-inducible promoter.

In some aspects, each of the polynucleotides of a single expression construct herein may include the same promoter so that each of the proteins encoded thereby is co-regulated or co-expressed. In some aspects, expression of each of the polynucleotides of a single expression construct herein may be regulated by the same inducible promoter so that each of the proteins encoded thereby is co-regulated or co-expressed. In some aspects, each of the polynucleotides of a single expression construct herein include different promoters so that a protein encoded by one polynucleotide may be expressed under different conditions than a protein encoded by another polynucleotide. In some aspects, each of the expression constructs herein include different promoters.

If not specified, it is to be understood that an expression construct herein includes sequences or elements necessary for the selection and/or maintenance of the expression cassette in a host cell. In some aspects, an expression includes at least one selection marker, an origin of replication, partition system, toxin-antitoxin system, and the like.

The term “selection marker” refers to a gene that aids in the selection of cells that actively express a nucleic acid sequence. Examples of suitable selection markers include enzymes that code for antibiotic resistance, such as kanamycin, ampicillin, neomycin, puromycin, hygromycin, blasticidin, or zeocin (i.e., antibiotic resistance genes).

Another example of a suitable selection marker is a fluorescent protein, such as green fluorescent protein (GFP), red fluorescent protein (RFP), or blue fluorescent protein (BFP). In some aspects, each of the expression constructs herein include a different selection marker, thereby allowing for the selection of host cells harboring each of the expression constructs.

The term “origin of replication” refers to a nucleic acid sequence at which replication of an expression construct herein is initiated. This allows the expression construct described herein to stably replicate and segregate with endogenous chromosomes in a suitable host cell (e.g., a microbial cell such as a bacterial or yeast cell) so that it may be transmitted to host cell progeny. In some aspects, the origin of replication is of bacterial origin. In some aspects, the origin of replication is a high copy number origin. In some aspects, the origin of replication may be derived from the pBR322 plasmid, pUC plasmid, pMB1 plasmid, ColE1 plasmid, R6K plasmid, p15A plasmid, pSC101 plasmid or F1 phagemid.

In a first expression construct of the system, kit herein and method herein, a SP6 promoter is operably linked to a polynucleotide encoding a first protein of interest. As is known in the art, SP6 is a bacteriophage that encodes a single-subunit RNA polymerase. SP6 promoters are composed of a highly conserved 17-bp sequence, ATTTAGGTGACACTATAG (SEQ ID NO:1), including two domains: an initiation domain downstream of −4 and a binding domain upstream of −5, which is bound by SP6 RNA polymerase. In some aspects, a first expression construct comprises a SP6 promoter comprising a sequence having at least 90% sequence identity to SEQ ID NO:1, e.g., a sequence having 90%, 91%, 92%, 93%, 94%, 95%, 96%, 97%, 98%, or 99% sequence identity to SEQ ID NO:1. See, e.g., Shin et al. (2000) Proc. Natl. Acad. Sci. USA 97(8):3890-3895 for examples of variant SP6 promoter sequences that bind SP6 RNA polymerase and initiate transcription.

A “polynucleotide encoding a first protein of interest” refers to a nucleotide sequence encoding a protein to be evolved in a continuous directed evolution process as described herein. A first protein of interest may be an enzyme, receptor, drug target, antibody, and the like. The first protein of interest may be evolved to improve, e.g., catalytic activity, transcriptional activation activity, bond-forming activity, stability, half-life, and/or binding activity. The term includes any variations of a protein of interest that are the result of a continuous evolution process according to methods described herein. For example, in some aspects, a first protein of interest is a protease to be evolved for increased stability at high temperatures and/or improved activity in the presence of detergents.

“Operably linked” or “operably associated,” as used herein in reference to polynucleotides, it is meant that the indicated elements are functionally related to each other and are also physically related. Thus, the term “operably linked” or “operably associated” as used herein, refers to nucleotide sequences on a single nucleic acid molecule that are functionally associated. Thus, a first polynucleotide sequence that is operably linked to a second polynucleotide sequence means a situation when the first polynucleotide sequence is placed in a functional relationship with the second polynucleotide sequence. For instance, a promoter is operably associated with a polynucleotide sequence if the promoter affects the transcription or expression of said polynucleotide sequence. Those skilled in the art will appreciate that the control sequences (e.g., promoter) need not be contiguous with the polynucleotide sequence to which it is operably associated, as long as the control sequences function to direct the expression thereof. Thus, for example, intervening untranslated, yet transcribed, nucleic acid sequences may be present between a promoter and the polynucleotide sequence, and the promoter may still be considered “operably linked” to the polynucleotide sequence.

In a second expression construct of the system, kit herein and method herein, at least one polynucleotide encoding a SP6 RNA polymerase-deaminase fusion protein is provided. A “fusion protein” refers to the coupling of two or more polypeptides/peptides so as to form a single contiguous protein. In some aspects, a fusion protein comprises two or more polypeptides/peptides that are covalently coupled in a single polypeptide chain. Covalently connected fusion proteins typically are produced genetically through the in-frame fusing of the polynucleotide sequences encoding for each of the said polypeptides/peptides. Expression of the fused coding sequence results in the generation of a single protein without any translational terminator between each of the polypeptides/peptides.

In some aspects, a SP6 RNA polymerase of use in the system, kit, and method herein has an amino acid that shares at least about 90% sequence identity to SEQ ID NO:2, e.g., about 90%, 91%, 92%, 93%, 94%, 95%, 96%, 97%, 98%, or 99% sequence identity to SEQ ID NO:2: MQDLHAIQLQLEEEMFNGGIRRFEADQQRQIAAGSESDTAWNRRLLSELIAPMAEGIQA YKEEYEGKKGRAPRALAFLQCVENEVAAYITMKVVMDMLNTDATLQAIAMSVAERIEDQ VRFSKLEGHAAKYFEKVKKSLKASRTKSYRHAHNVAVVAEKSVAEKDADFDRWEAWPKE TQLQIGTTLLEILEGSVFYNGEPVFMRAMRTYGGKTIYYLQTSESVGQWISAFKEHVAQ LSPAYAPCVIPPRPWRTPFNGGFHTEKVASRIRLVKGNREHVRKLTQKQMPKVYKAINA LQNTQWQINKDVLAVIEEVIRLDLGYGVPSFKPLIDKENKPANPVPVEFQHLRGRELKE MLSPEQWQQFINWKGECARLYTAETKRGSKSAAVVRMVGQARKYSAFESIYFVYAMDSR SRVYVQSSTLSPQSNDLGKALLRFTEGRPVNGVEALKWFCINGANLWGWDKKTFDVRVS NVLDEEFQDMCRDIAADPLTFTQWAKADAPYEFLAWCFEYAQYLDLVDEGRADEFRTHL PVHQDGSCSGIQHYSAMLRDEVGAKAVNLKPSDAPQDIYGAVAQVVIKKNALYMDADDA TTFTSGSVTLSGTELRAMASAWDSIGITRSLTKKPVMTLPYGSTRLTCRESVIDYIVDL EEKEAQKAVAEGRTANKVHPFEDDRQDYLTPGAAYNYMTALIWPSISEVVKAPIVAMKM IRQLARFAAKRNEGLMYTLPTGFILEQKIMATEMLRVRTCLMGDIKMSLQVETDIVDEA AMMGAAAPNFVHGHDASHLILTVCELVDKGVTSIAVIHDSFGTHADNTLTLRVALKGQM VAMYIDGNALQKLLEEHEERWMVDTGIEVPEQGEFDLNEIMDSEYVFA (SEQ ID NO:2). In some aspects, a SP6 RNA polymerase of use in the system, kit, and method described herein binds to the SP6 promoter (SEQ ID NO:1) and initiates transcription of the polynucleotide encoding the first protein of interest to produce said protein of interest.

“Deaminase” refers to an enzyme that catalyzes a deamination reaction. In some aspects, a deaminase refers to a cytosine deaminase that catalyzes the deamination of cytidine or deoxycytidine to uracil or deoxyuracil, respectively. In some aspects, a deaminase refers to adenine deaminase which is capable of catalyzing the formation of inosine (I) by adenosine or deoxyadenosine (A).

In one aspect, a fusion protein herein is composed of a SP6 RNA polymerase and a cytosine deaminase. Cytosine deaminase proteins have been identified in various species and are known to those of skill in the art. For example, cytosine deaminase may include, but are not limited to, APOBEC1, APOBEC3B, CDA1, AID, and evoFERNY. See paragraphs [0487] and [0496]-[0502] of US 2022/0315906, incorporated herein for its disclosure of sequences of cytosine deaminases. In some aspects, the SP6 RNA polymerase-cytosine deaminase fusion protein includes the cytosine deaminase from Petromyzon marinus, known under GENBANK Accession No. ABO15149.1. In some aspects, the SP6 RNA polymerase is C-terminal to the cytosine deaminase. In some aspects, the SP6 RNA polymerase is N-terminal to the cytosine deaminase.

In another aspect, a fusion protein herein is composed of a SP6 RNA polymerase and an adenine deaminase. Adenine deaminase proteins have been identified in various species and are known to those of skill in the art. For example, human genes encoding a TadA protein include ADAT1 and ADAT2. E. coli TadA, TadA7.10 dimer, TadA8e, TadA9 and mouse ADA are additional examples. See paragraphs [0435]-[0438] of US 2022/0315906, incorporated herein for its disclosure of sequences of cytosine deaminases. In some aspects, the SP6 RNA polymerase-adenine deaminase fusion protein includes the adenine deaminase TadA8e, known under GENBANK Accession No. UNJ19119.1 and PDB accession 6VPC E. In some aspects, the SP6 RNA polymerase is C-terminal to the adenine deaminase. In some aspects, the SP6 RNA polymerase is N-terminal to the adenine deaminase.

In some aspects, a second expression construct herein includes at least two deaminases. In some aspects, the fusion protein comprises more than one deaminase fused to an SP6 RNA polymerase, which may be arranged in any manner. For example, a fusion protein comprising two deaminases (“D”) fused to one SP6 RNA polymerase (“P”) may be structured from N-terminus to C-terminus as follows: (i) D-P-D; (ii) D-D-P; or (iii) P-D-D. In some aspects, when at least two deaminases are fused to an SP6 RNA polymerase, at least one deaminase is an adenine deaminase and the other deaminase is a cytosine deaminase.

In some aspects, the second expression construct comprises a polynucleotide encoding a SP6 RNA polymerase-cytosine deaminase fusion protein and a polynucleotide encoding a SP6 RNA polymerase-adenine deaminase fusion protein.

In one or more the fusion proteins herein, the polypeptides/peptides of the fusion proteins may be directly fused together or the polypeptides/peptides of the fusion proteins may be fused to one through a linker. As used herein, the term “linker” refers to a flexible molecule used to connect two molecules of interest together. In some aspects, the linker is a peptide linker. In some aspects, the peptide linker is an XTEN linker (Schellenberger et a. (2009) Nat. Biotechnol. 27(12):1186-90) or a Gly-Ser linker.

In some aspects, the first expression construct and/or second expression construct herein further comprises a polynucleotide encoding a DNA repair inhibitor. In some aspects, the DNA repair inhibitor is a uracil DNA glycosylase (UNG) inhibitor peptide (UGI). An exemplary UGI is provided under GENBANK Accession No. P14739.1.

As indicated, the flexibility of the system herein may be extended to include the expression of a chaperone protein, accessory protein, and the like, which may facilitate mutation of the first protein of interest. Accordingly, in some aspects, the system, kit, and method may further include a polynucleotide encoding a second protein of interest operably linked to a T7 promoter. The T7 promoter is well known in the art and may include the sequence TAATACGACTCACTATAG (SEQ ID NO:3). In some aspects, the second protein of interest is a chaperone protein such as GroEL/GroES or Cpn20/Cpn60. In some aspects, the second protein of interest is an accessory protein such as RbcX, Raf1, Raf2, BSD2, or rubisco activase. In some aspects, the T7 promoter operably linked to a polynucleotide encoding a second protein of interest may be a component of the first expression construct and/or second expression construct. In other aspects, the T7 promoter operably linked to a polynucleotide encoding a second protein of interest may be a component is provided as a component of a third expression construct.

A kit for performing continuous directed evolution is also provided. A kit herein includes (a) a polynucleotide encoding a SP6 RNA polymerase, (b) a polynucleotide encoding a cytosine deaminase and/or, a polynucleotide encoding a adenine deaminase, and (c) a SP6 promoter. In some aspects, the kit may further include a polynucleotide encoding a DNA repair inhibitor. In some aspects, the kit may further include a T7 promoter. In some aspects, at least one component in the kit is provided in a desiccated or lyophilized form. In other aspects, at least one component of the kit is provided in a solubilized form. In some aspects, the kit further comprises at least one buffer. In some aspects, at least one of the buffers is a reaction buffer.

Expression constructs may be prepared by any conventional method. For example, the individual components may be assembled using overlap-extension polymerase chain reaction (PCR), polymerase cycling assembly, sticky end ligation, BioBrick assembly, Golden Gate assembly, Gibson assembly, recombinase assembly, ligase cycling reaction, or template directed ligation. In some aspects, expression constructs herein are prepared by Golden Gate assembly. As is known in the art, Golden Gate uses type IIS restriction enzymes for single-step, ordered, multipart assembly. Accordingly, in some aspects, the components of the kit herein are compatible with Golden Gate assembly and include appropriate 3′ and 5′ overhangs and are devoid of type IIS restriction enzyme recognitions sites, e.g., devoid of recognition sites for BbsI, BsaI and SapI. See, e.g., the Examples herein.

In some aspects, the system, kit, and method herein provide for host cells. In some aspects, the host cells may be cyanobacterial cells, mammalian cells, fungal cells (e.g., yeast cells), or bacterial cells. For example, the host cells may be E. coli cells such as TOP10, OMNIMAX™, PIR1, PIR2, INVaF, INV110, BL21, Machl, DHl0Bac, DH10B, DH12S, DH5a, Stbl2, Stbl3, Stbl4, XL1-Blue, XL2-Blue, and related strains.

A method of continuous directed evolution of a protein of interest is also provided, which includes the steps of introducing into a host cell a first expression construct comprising a SP6 promoter operably linked to a polynucleotide encoding the protein of interest; introducing into the host cell a second expression construct comprising a polynucleotide encoding a SP6 RNA polymerase-cytosine deaminase fusion protein and a polynucleotide encoding a SP6 RNA polymerase-adenine deaminase fusion protein; and culturing the host cell under conditions suitable for continuous directed evolution of the protein of interest. The second expression construct comprises a polynucleotide encoding a SP6 RNA polymerase-cytosine deaminase fusion protein and a polynucleotide encoding a SP6 RNA polymerase-adenine deaminase fusion protein. As used herein, the term “introducing” or “transforming” means presenting an expression construct to the host cell in such a manner that the expression construct gains access to the interior of the host cell.

Culturing the host cell “under conditions suitable for continuous directed evolution” of the protein of interest refers to growing the host cell under conditions to allow for multiple rounds of (a) replication the first expression construct, (b) mutation of polynucleotide encoding the protein of interest, and (c) selection to produce a desired evolved protein of interest.

The following examples are provided for illustrative purposes only and not intended to limit the scope of the claims.

### EXAMPLES

### Example 1: Materials and Methods

Strains and growth conditions. For cloning, E. coli 5a (NEB) was grown in liquid Terrific Broth (TB) and selected on Lysogeny broth (LB) agar 1.5% (w/v) plates, supplemented with appropriate antibiotics (kanamycin, 50 μg/mL, spectinomycin, 100 μg/mL, ampicillin, 100 μg/mL, chloramphenicol, 34 μg/mL). For evolution experiments, E. coli BL21 (NEB) or 5a (NEB) were plated in LB agar supplemented with 100 μg/mL ampicillin, and 50 μg/mL kanamycin or 34 μg/mL chloramphenicol, and 0.2% arabinose to allow for expression.

Assembly of Plasmids. Oligonucleotides were purchased from Twist Bioscience, GenScript and Integrated DNA Technologies (Table 1). Parts were domesticated, removing internal BbsI, BsaI and SapI restriction sites by PCR with Q5 (NEB) using overhang primers, and cloned into the universal acceptor vector, pLOR. Spectinomycin resistance artificial stop codon mutants were created by inverse PCR followed by Gibson Assembly (Gibson et al. (2009) Nat. Methods 6(5):343-5; Olszakier et al. (2022) BMC Biotechnol. 22:10).

TABLE 1

| | | |
|---|---|---|
| | | SEQ ID |
| Primer name | Sequence (5′ -> 3′) | NO: |

Primers for L0 T7mut2 cloning using T7mut2 synthesized gene
fragment as template

| | | |
|---|---|---|
| F.T7mut2.GGAG.pL0R | AAGCTCTTCATCCGGAGAATTCTAATACGACT | 4 |
| R.T7mut2.AGGT.pL0R | TTGCTCTTCTTCGACCTTTTCTCCTCTTTTCT | 5 |
| R.T7mut2.AATG.pL0R | TTGCTCTTCTTCGcatTTTTCTCCTCTTTTCTCT | 6 |
| | CTC | |

Primers for L0 specR cloning to CDS2 overhang using pC0.028¹ as
template

| | | |
|---|---|---|
| F.specR.AGGT.pL0R | AAGCTCTTCATCCAGGTATGCGCGAAGCGGTT | 7 |
| R.specR.GCTT.pL0R | TTGCTCTTCTTCGAAGCTTACTTGCCGACCAC | 8 |

Primers for L0 specR mutant cloning.In brief, for each stop
codon, PCRs B and A were set up using the corresponding stop
codon primers and the corresponding general Gibson primers.
They were then purified and assembled through Gibson reaction.
pIM004 was used as template

| | | |
|---|---|---|
| F.Gibson.B.specR | tctgcgctctgctgaagccagttaccttcggaaa | 9 |
| | aagagt | |
| B.reverse.pLOr. | gtactcacttcagcaataaccgcttagcgcatac | 10 |
| specR.E3* | ctCGAG | |
| B.reverse.pLOr. | aggtcaatatcgctatgtggctacaagcccccat | 11 |
| specR.K43* | cgaccg | |
| B.reverse.pLOr. | caataacaccgaccacttcagattactgggtact | 12 |
| specR.L13* | cacttcagc | |
| B.reverse.pLOr. | catcgaccgcggaaccatactaatgtaccgctag | 13 |
| specR.L33* | taaagt | |
| B.reverse.pLOr. | cttcagcaataaccgcttcgcggatacctCGAGA | 14 |
| specR.MII.C* | CCAGAagaccGAATTC | |
| B.reverse.pLOr. | cttcagcaataaccgcttcgcgcagacctCGAGA | 15 |
| specR.M1L | CCAGAagaccGAATTC | |
| B.reverse.pLOr. | cttcagcaataaccgcttcgcgcgtacctCGAGA | 16 |
| specR.MIT | CCAGAagaccGAATTC | |
| B.reverse.pLOr. | caccgaccacttcagataactaggtactcacttc | 17 |
| specR.Q12* | agcaat | |
| B.reverse.pLOr. | tcaagcccccatcgaccgcttaaccatacaaatg | 18 |
| specR.S36* | taccgc | |
| B.reverse.pLOr. | cgcgtttagccggataccgctaaggaatgatatc | 19 |
| specR.W99* | atcatg | |
| R.Gibson.A.specR | actctttttccgaaggtaactggcttcagcagag | 20 |
| | cgcaga | |
| A.forward.pLOr. | CTCGaggtatgcgctaagcggttattgctgaagt | 21 |
| specR.E3* | gagtac | |
| A.forward.pLOr. | cggtcgatgggggcttgtagccacatagcgatat | 22 |
| specR.K43* | tgacct | |
| A.forward.pLOr. | gctgaagtgagtacccagtaatctgaagtggtcg | 23 |
| specR.L13* | gtgttattg | |
| A.forward.pLOr. | actttactagcggtacattagtatggttccgcgg | 24 |
| specR.L33* | tcgatg | |
| A.forward.pLOr. | GAATTCggtctTCTGGTCTCGaggtatccgcgaa | 25 |
| specR.M1I.C* | gcggttattgctgaag | |
| A.forward.pLOr. | GAATTCggtctTCTGGTCTCGaggtctgcgcgaa | 26 |
| specR.M1L | gcggttattgctgaag | |
| A.forward.pLOr. | GAATTCggtctTCTGGTCTCGaggtacgcgcgaa | 27 |
| specR.MIT | gcggttattgctgaag | |
| A.forward.pLOr. | attgctgaagtgagtacctagttatctgaagtgg | 28 |
| specR.Q12* | tcggtg | |
| A.forward.pLOr. | gcggtacatttgtatggttaagcggtcgatgggg | 29 |
| specR.S36* | gcttga | |
| A.forward.pLOr. | catgatgatatcattccttagcggtatccggcta | 30 |
| specR.W99* | aacgcg | |

Primers for L0 SP62mut2 cloning with SP6mut2 synthesized gene
fragment as template

| | | |
|---|---|---|
| F.sp6mut2.GGAG. | AAGCTCTTCATCCGGAGAATTCATT | 31 |
| pL0R | | |
| R.sp6mut2.AGGT. | TTGCTCTTCTTCGACCTTTTCTCCT | 32 |
| pL0R | | |

Primers for L0 UGI cloning with pHyo094² as template

| | | |
|---|---|---|
| F.UGI.AATG.pL0R | AAGCTCTTCATCCaatgaccaac | 33 |
| | ctttccgacat | |
| R.UGI.GCTT.pL0R | TTGCTCTTCTTCGaagcttacag | 34 |
| | catcttgatcttgtt | |

Primers for L0 TadASE cloning with pDae079³ as template

| | | |
|---|---|---|
| F.TadA8E.AATG.pL0R | AAGCTCTTCATCCaatgagtgaagttgaattcag | 35 |
| | ccatgaatattggatgc | |
| R.TadA8E.AGGT.pL0R | TTGCTCTTCTTCGacctaaactt | 36 |
| | tcgggtgtggcggac | |

Primers for domestication of pBAD and araC pBAD operon with
pC0.0931 or pDae079³ as template

| | | |
|---|---|---|
| F.pBAD.dom.F1F | AAGCTCTTCATCCggagaagaaa | 37 |
| | ccaattgtccatattgcatc | |
| R.pBAD.dom.F1R | AAGCTCTTCAtgccagtaaaagacgcagt | 38 |
| F.pBAD.dom.F2F | AAGCTCTTCAgcacttctcgctaaccaaac | 39 |
| F.pBAD.dom.F2R | TTGCTCTTCTTCGcatttatggag | 40 |
| | aaacagtagagagttgc | |
| F.araC- | AAGCTCTTCATCCggagcaaatgga | 41 |
| pBAD.dom.F1F | cgaagcagggat | |
| R.araC- | AAGCTCTTCAtgccagtaaaagacgcagt | 42 |
| pBAD.dom.F1R | | |
| F.araC- | AAGCTCTTCAgcacttctcgctaaccaaac | 43 |
| pBAD.dom.F2F | | |
| F.araC- | TTGCTCTTCTTCGcatttgccat | 44 |
| pBAD.dom.F2R | ggtgaattcctcc | |

Primers for domestication of T7 RNAP (BpiI free) with pIM021 as
template

| | | |
|---|---|---|
| F.T7RNAP.dom.F1F | AAGCTCTTCActccggcttcatcggtagtt | 45 |
| F.T7RNAP.dom.F1R | AAGCTCTTCAgtccggcttcatcggtagtt | 46 |
| F.T7RNAP.dom.F2F | AAGCTCTTCAgaggacatcgatatgaacccc | 47 |
| F.T7RNAP.dom.F2R | AAGCTCTTCAgtatcttccaggacttgttgc | 48 |
| F.T7RNAP.dom.F3F | AAGCTCTTCAtaccattcagcctgcgatcg | 49 |
| F.T7RNAP.dom.F3R | TTGCTCTTCTTCGaagcttaggcgaatgcgaa | 50 |

Primers for L0 rhaB cloning with pSIJ8⁴ as template

| | | |
|---|---|---|
| F.rhaB.GGAG.pL0R | AAGCTCTTCATCCGGAGctggctaa | 51 |
| | atacggaaggaa | |
| R.rhaB.AATG.pL0R | TTGCTCTTCTTCGCATTctagtattt | 52 |
| | ctcctcaatttcatta | |

Primers for L0 cloning of T7 RNAP with alternative codon usage
with pDae079³ as template

| | | |
|---|---|---|
| F.T7RNAP.Dae.AGGT. | AAGCTCTTCATCCaggtaacacgatt | 53 |
| pL0R | aacatcgctaagaac | |
| R.T7RNAP.Dae.GCTT. | TTGCTCTTCTTCGAAGCttacgcgaa | 54 |
| pL0R | cgcgaagtc | |
| F.T7RNAP.Dae.AATG. | AAGCTCTTCATCCaatgaacacgat | 55 |
| pL0R | taacatcgctaagaac | |

Primers for cloning of compact, non-repetitive spacers

| | | |
|---|---|---|
| F.new.spacerOdd 1 | GCATGGTCTCAggagCCTATT | 56 |
| | CTGGTGGAACTGGATG | |
| F.new.spacerOdd 2 | GCATGGTCTCAggagGAAGGCGAT | 57 |
| | ACCCTGGTAAAC | |
| F.new.spacerOdd 3 | GCATGGTCTCAggagCGCAAAGCG | 58 |
| | TTCTGTCTAAAG | |
| F.new.spacerOdd 4 | GCATGGTCTCAggagGCTGACCCT | 59 |
| | GAAGTTCATCT | |
| F.new.spacerEven 1 | GCATGCTCTTCAatgCAAGGAGGACGGCAACAT | 60 |
| F.new.spacerEven 2 | GCATGCTCTTCAatgGAGTTCGA | 61 |
| | AATCGAAGGTGAAG | |
| F.new.spacerEven 3 | GCATGCTCTTCAatgCGAGTGAAA | 62 |
| | CACAGGAAGATCA | |
| F.new.spacerEven 4 | GCATGCTCTTCAatgCTGCGCCAA | 63 |
| | TTGATGACAATAC | |
| R.new.spacerOdd 1 | GCATGGTCTCAagcgTGCTTCATA | 64 |
| | TGGTCCGGATAAC | |
| R.new.spacerOdd 2 | GCATGGTCTCAagcgAACAGGACC | 65 |
| | ATCACCGATTG | |
| R.new.spacerOdd 3 | GCATGGTCTCAagcgTGAGCCAGT | 66 |
| | GTGACTCTAGTA | |
| R.new.spacerOdd 4 | GCATGGTCTCAagcgGTCTTGTAG | 67 |
| | TTGCCGTCGT | |
| R.new.spacerEven 1 | GCATGCTCTTCAaccGGACTGGTA | 68 |
| | GCTCAGGTAGT | |
| R.new.spacerEven 2 | GCATGCTCTTCAaccCGTTCCCA | 69 |
| | TTTGAAACCTTCC | |
| R.new.spacerEven 3 | GCATGCTCTTCAaccACCGTTGC | 70 |
| | CCTTCTCAATAC | |
| R.new.spacerEven 4 | GCATGCTCTTCAaccCTGAAACC | 71 |
| | ACAGCCACATTAAC | |

Primers for extracting gene of interest from an
evolving/evolved population, where N₇ are multiplexing barcodes
with Any pJUMP27 plasmid as template

| | | |
|---|---|---|
| F.JUMP27.GOI.seq | aNNNNNNNctcgggcaaatcgctgaata | 72 |
| R.JUMP27.GOI.seq | tNNNNNNNcttgtgcaatgtaacatcagagatt | 73 |

¹Vasudevan et al.(2019) Plant Physiol. 180(1): 39-55.
²Park & Kim (2021) Nucleic Acids Res. 49(6): e32.
³Seo et al.(2023) Nucleic Acids Res. 51(10): e59.
⁴Jensen et al.(2015) Sci.Rep. 5: 17874.

Plasmids were generated by Golden Gate Assembly, following plant modular cloning syntax (Engler et al. (2014) ACS Synth. Biol. 3(11):839-843; Vasudevan et al. (2019) Plant Physiol. 180(1):39-55; Werner et al. (2012) Bioengineered 3(1):38-43; Weber et al. (2011) PLOS ONE 6(2):e16765) and using the Universal Loop assembly system (Pollak et al. (2019) New Phytol. 222(1):628-40; Pollak et al. (2020) Synth. Biol. 5(1):ysaa001). Briefly, a mixture of 1.5 nM of each insert and 0.75 nM backbone was assembled with 0.5 U/μL BsaI-HF-v2 (NEB) or 0.5 U/μL SapI (NEB) and 10 U/μL T4 DNA ligase (NEB) in 1× T4 DNA ligase buffer and BSA (0.05 mg/mL, for BsaI reactions) or 1× rCutSmart (for SapI reactions). Reactions were cycled 25 times for 37° C. for 3 minutes, 16° C. for 4 minutes, then 50° C. for 20 minutes and 80° C. for 20 minutes. The plasmid product was transformed into E. coli, extracted with ZymoPure kit according to the manufacturer's instruction, and analyzed by whole plasmid sequencing (Plasmidsaurus). Plasmids used are provided in Table 2.

TABLE 2

| Plasmid | Origin of | | | |
|---|---|---|---|---|
| name | replication | Description | Level | Notes |
| pHyo094¹ | p15A | araC_pBAD_PmCDA1:T7RNAP - | NA | eMutaT7 |
| | | UGI (marker: Chloramphenicol) | | |
| pDae079² | p15A | araC_pBAD_PmCDA1:T7RNAP -- | NA | eMutaT7 |
| | | pBAD_TadA8E:T7RNAP - UGI | | transition |
| | | (marker: Chloramphenicol) | | |
| pIM315 | pSC101 (non- | T7mut2_SpecR(L13*)_{—} | 1 | Target plasmid |
| | temperature | TrrnB (marker: Ampicillin) | | for specR stop |
| | sensitive) | | | codon evolution - |
| | | | | T7 responsive |
| | | | | promoter |
| pIM316 | pSC101 (non- | SP6mut2_specR(L13*)_{—} | 1 | Target plasmid |
| | temperature | TrrnB (marker: Ampicillin) | | for specR stop |
| | sensitive) | | | codon evolution - |
| | | | | SP6 responsive |
| | | | | promoter |
| pIM375 | p15A | araC_pBAD_PmCAID:T7RNAP -- | 3 | eMutaT7 |
| | | pBAD_TadA8E:T7RNAP - | | transition |
| | | odd spacer 3 -- J23104_UGI | | reassembled - |
| | | (marker: Kanamycin) | | p15 backbone |
| pIM372 | pBR322 | araC_pBAD_PmCAID:T7RNAP -- | 3 | eMutaT7 |
| | | pBAD_TadA8E:T7RNAP - | | transition |
| | | odd spacer 3 - J23104_UGI | | reassembled -- |
| | | (marker: Kanamycin) | | pCA backbone |
| pIM376 | p15A | araC_pBAD_PmCAID:SP6RNAP -- | 3 | eMutaSP6 - pCA |
| | | pBAD_TadA8E:SP6RNAP -- | | backbone |
| | | odd spacer 3 -- J23104_UGI | | |
| | | (marker: Kanamycin) | | |
| pIM378 | p15A | araC_pBAD_SP6RNAP | 1 | eMutaT7 (assembled) |
| | | (marker: Kanamycin) | | p15 background |

¹Park & Kim (2021) Nucleic Acids Res. 49 (6): e32.
²Seo et al. (2023) Nucleic Acids Res. 51 (10): e59.

Evolution experiments. Colonies were picked into 3 mL LB with kanamycin and ampicillin and grown to saturation. This was passage 0 (P0). This culture was diluted 1:1000 in 3 mL of fresh LB supplemented with 0.2% 0.22-μm-filter-sterilized arabinose, kanamycin, and ampicillin, and grown to saturation (approximately 12 hours). This was a single passage and repeated up to 14 times. At the end of each passage, serial dilutions were made and plated in selective (spectinomycin 100 μg/mL) and non-selective media.

Suppressor frequency was calculated as colony forming units (cfu) per mL (selective) over cfu/mL (non-selective) as described (Seo et al. (2023) Nucleic Acids Res. 51(10):e59). Populations that showed no colonies in selective plates were graphed as log₁₀(suppressor frequency)=−9 for visualization purposes. To determine mutation count, colonies were picked, then grown in LB with spectinomycin (100 μg/mL), plasmids extracted, and target sequences amplified by PCR using primers F.JUMP27.GOI.seq and R.JUMP27.GOI.seq (Table 1), multiplexed, and sequenced by Plasmidsaurus Premium PCR. The sequences were analyzed with a simplified MAPLE pipeline (Rix et al. (2024) Science 386(6722):eadm9073) and aligned to the unmutated plasmid sequence. Mutations in the sequence were counted if appearing at frequencies over 0.5 in the samples.

Golden Gate Reaction Protocol. The volumes in Table 3 are per reaction volumes. Master mixes were made for one extra reaction to account for pipetting inaccuracies. Master mixes were briefly vortexed and spun prior to use.

TABLE 3

| | | Volume | | | Volume |
|---|---|---|---|---|---|
| Reagent | Conc. | (μL) | Reagent | Conc. | (μL) |
| HPLC H20 | | 3.5 | HPLC H20 | | 3 |
| Cutsmart | 10X | 0.5 | T4 DNA ligase | 10X | 1 |
| buffer | | | buffer | | |
| T4 DNA | 10X | 0.5 | Diluted BSA | 1 | 0.5 |
| ligase | | | (Bovine Serum | mg/mL | |
| buffer | | | Albumin) | | |
| T4 DNA | 400 | 0.25 | T4 DNA | 400 | 0.25 |
| ligase | u/μL | | ligase | U/μL | |
| SapI | 10 | 0.25 | BsaI (or | 10 | 0.25 |
| | U/μL | | BsaI-HFv2) | U/μL | |

Each part-containing plasmid was quantified for DNA concentration with Nanodrop. Subsequently, each plasmid was diluted in MilliQ water to 15 fmol/μL, while dilution was to 7.5 fmol/μL for receiver plasmids. This is equivalent in ng/μL to length of the plasmid (in bp) divided by 100 for inserts or by 200 for receivers. All remaining steps were carried out on ice. For each reaction, 1 μL of each diluted plasmid required for it was combined in a PCR tube and gently mixed. For reactions with fewer than 5 total plasmids, MilliQ water was added to a total volume of 5 μL. Reaction mixes containing the required enzymes and buffers were prepared as above. To each reaction, 5 μL of reaction mix was added for a total of 10 μL, briefly vortexed and spun.

The assembled reaction mixes were transferred to a thermocycler and underwent the following program: 37° C. for 3 minutes, then 16° C. for 4 minutes. This was repeated for a minimum of 24×. Then, 50° C. for 20 minutes, and 80° C. for 20 minutes. The reaction was then cooled at 4° C. indefinitely and then transformed. This protocol was adapted from Loop and uLoop assembly V5. It was observed that the prolonged final digestion step led to fewer undigested backbone vectors.

Statistical analysis. Data was processed and analyzed in R 4.4.0 and RStudio 2024.12.0+467. Graphs were made through ggplot2, Viridis, and ggpubR. Data was checked for normality by Shapiro-Wilk test.

### Example 2: Dedicated Continuous Directed Evolution Toolkit

A toolkit was created by separating the components of eMutaT7^{transition} into level 0 parts, in addition to a T7-responsive promoter, T7ₘᵤₜ₂, containing an optimized ribosome binding sequence (Jin et al. (2019) ACS Synth. Biol. 8 (4):655-60), inducible promoters, cytosine and adenine deaminases, and variants of the coding sequences that connect with and without the need for deaminases, as control parts (Table 4). Because T7 RNA polymerase (RNAP) is used as an expression system, continuous directed evolution with MutaT7-style tools is incompatible when T7 is used for the expression of other genes, which could be important when chaperones or other heterologous proteins need to be expressed to enable evolution schemes. To address this, SP6 RNAP from SP6 phage was used. An SP6-specific promoter, SP6ₘᵤₜ₂, was created wherein the T7 binding sequence was modified from T7mut2 for recognition by SP6.

TABLE 4

| | | | Overhang | | |
|---|---|---|---|---|---|
| Plasmid name | ORI | Description | 5′ | 3′ | Notes |
| pIM004 | pBR322 | L0M-CDS-aadA | AGGT | GCTT | Spectinomycin |
| | | (marker: | | | resistance |
| | | Spectinomycin) | | | gene |
| pIM005¹ | pBR322 | L0M-P-T7mut2 | GGAG | AGGT | T7 responsive |
| | | (marker: | | | promoter with |
| | | Spectinomycin) | | | optimized RBS |
| pIM007¹ | pBR322 | L0M-P-T7mut2 | GGAG | AATG | T7 responsive |
| | | (marker: | | | promoter with |
| | | Spectinomycin) | | | optimized RBS |
| pIM008 | pBR322 | L0M-CDS2-aadA | AGGT | GCTT | Spectinomycin |
| | | (L33*) (marker: | | | mutant |
| | | Spectinomycin) | | | library. |
| pIM009 | pBR322 | L0M-CDS2-aadA | AGGT | GCTT | Note: used |
| | | (K43*) (marker: | | | CDS2 syntax |
| | | Spectinomycin) | | | to allow for |
| pIM010 | pBR322 | L0M-CDS2-aadA | AGGT | GCTT | removal of |
| | | (W99*) (marker: | | | start codon |
| | | Spectinomycin) | | | and should |
| pIM011 | pBR322 | L0M-CDS2-aadA | AGGT | GCTT | use the |
| | | (L13*) (marker: | | | promoter |
| | | Spectinomycin) | | | version that |
| pIM013 | pBR322 | L0M-CDS2-aadA | AGGT | GCTT | enables |
| | | (S36*) (marker: | | | assembly. |
| | | Spectinomycin) | | | However, |
| pIM017 | pBR322 | L0M-CDS2-aadA | AGGT | GCTT | start codon |
| | | (Q12*) (marker: | | | mutants did |
| | | Spectinomycin) | | | not show |
| | | | | | growth |
| | | | | | inhibition. |
| pIM018 | pBR322 | L0M-CDS-T7RNAP | AGGT | GCTT | T7 RNA |
| | | (marker: | | | polymerase |
| | | Spectinomycin) | | | (codon |
| | | | | | optimized) |
| pIM019 | pBR322 | L0M-CDS-SP6RNAP | AATG | GCTT | SP6 RNA |
| | | (marker: | | | Polymerase |
| | | Spectinomycin) | | | |
| pIM020² | pTwist- | L0M-SP-PmCAID | AGGT | AATG | Petromyzon |
| | Amp | (marker: | | | marinus |
| | | Ampicillin) | | | cytidine |
| | | | | | deaminase |
| | | | | | (PmCDA1) |
| pIM021 | pTwist- | L0M-CDS2-T7RNAP | AGGT | GCTT | T7 RNA |
| | Amp | (marker: | | | polymerase |
| | | Ampicillin) | | | (codon |
| | | | | | optimized) |
| pIM022 | pTwist- | L0M-CDS2- | AGGT | GCTT | SP6 RNA |
| | Amp | SP 6RNAP | | | Polymerase |
| | | (marker: | | | |
| | | Ampicillin) | | | |
| pIM029 | pBR322 | L0M-P-SP6mut2 | GGAG | AATG | SP6 |
| | | (marker: | | | responsive |
| | | Spectinomycin) | | | promoter with |
| | | | | | optimized RBS |
| pIM030³ | pBR322 | L0M-SP-TadA8e | AATG | AGGT | Adenosine |
| | | (marker: | | | deaminase |
| | | Spectinomycin) | | | TadA8E |
| pIM031² | pBR322 | L0M-CDS-UGI | AATG | GCTT | Uracil-N- |
| | | (marker: | | | glycosylase |
| | | Spectinomycin) | | | inhibitor |
| pIM047 | pBR322 | L0M-P-SP6mut2 | GGAG | AGGT | SP6 |
| | | (marker: | | | responsive |
| | | Spectinomycin) | | | promoter with |
| | | | | | optimized RBS |
| pIM157 | pBR322 | L0M-CDS2-T7RNAP | AGGT | GCTT | T7 RNA |
| | | (marker: | | | polymerase |
| | | Spectinomycin) | | | (codon |
| | | | | | optimized, |
| | | | | | domesticated |
| | | | | | from BpiI |
| | | | | | sites) |
| pIM308⁴ | pBR322 | L0M-P-Prha | GGAG | AATG | Rhamnose |
| | | (marker: | | | inducible |
| | | Spectinomycin) | | | operon |
| pIM344² | pBR322 | L0M-CDS-T7RNAP | AATG | GCTT | T7 RNA |
| | | (marker: | | | polymerase |
| | | Spectinomycin) | | | |
| pIM359⁵ | pBR322 | L0M-P-Pbad | GGAG | GCTT | Arabinose |
| | | (marker: | | | responsive |
| | | Spectinomycin) | | | promoter |
| | | | | | (SapI |
| | | | | | domesticated) |
| pIM360² | pBR322 | L0M-P-araC: Pbad | GGAG | AATG | Arabinose |
| | | (marker: | | | responsive |
| | | Spectinomycin) | | | promoter, |
| | | | | | including |
| | | | | | araC |
| | | | | | repressor |
| | | | | | (SapI |
| | | | | | domesticated) |
| pIM345² | pBR322 | L0M-CDS2-T7RNAP | AGGT | GCTT | T7 RNA |
| | | (marker: | | | polymerase |
| | | Spectinomycin) | | | |
| Odd Spacer 1 | pCAo-1 | Odd Spacer 1 | ATG | GCA | sfGFP 211 bp |
| | | (marker: | | | fragment |
| | | Kanamycin) | | | |
| Odd Spacer 2 | pCAo-2 | Odd Spacer 2 | GCA | TAC | sfGFP 250 bp |
| | | (marker: | | | fragment |
| | | Kanamycin) | | | |
| Odd Spacer 3 | pCAo-3 | Odd Spacer 3 | TAC | CAG | pHluorin2, |
| | | (marker: | | | rrnBT1 219 bp |
| | | Kanamycin) | | | fragment |
| Odd Spacer 4 | pCAo-4 | Odd Spacer 4 | CAG | GGT | mVenus 212 bp |
| | | (marker: | | | fragment |
| | | Kanamycin) | | | |
| Even Spacer 1 | pCAe-1 | Even Spacer 1 | GGAG | TACT | sfGFP 232 bp |
| | | (marker: | | | fragment |
| | | Spectinomycin) | | | |
| Even Spacer 2 | pCAe-2 | Even Spacer 2 | TACT | AATG | mRFP 216 bp |
| | | (marker: | | | fragment |
| | | Spectinomycin) | | | |
| Even Spacer 3 | pCAe-3 | Even Spacer | AATG | GCTT | GEA2 222 bp |
| | | 3 (marker: | | | fragment |
| | | Spectinomycin) | | | |
| Even Spacer 4 | pCAe-4 | Even Spacer 4 | GCTT | CGCT | Derived from |
| | | (marker: | | | PYTK074 |
| | | Spectinomycin) | | | |

ORI, origin of replication.
¹Jin et al.(2019) ACS Synth. Biol. 8(4): 655-60.
²Park & Kim (2021) Nucleic Acids Res. 49(6): e32.
³Seo et al.(2023) Nucleic Acids Res. 51(10): e59.
⁴Jensen et al.(2015) Sci. Rep. 5: 17874.
⁵Derived from Vasudevan et al. (2019) Plant Physiol. 180(1): 39-55.

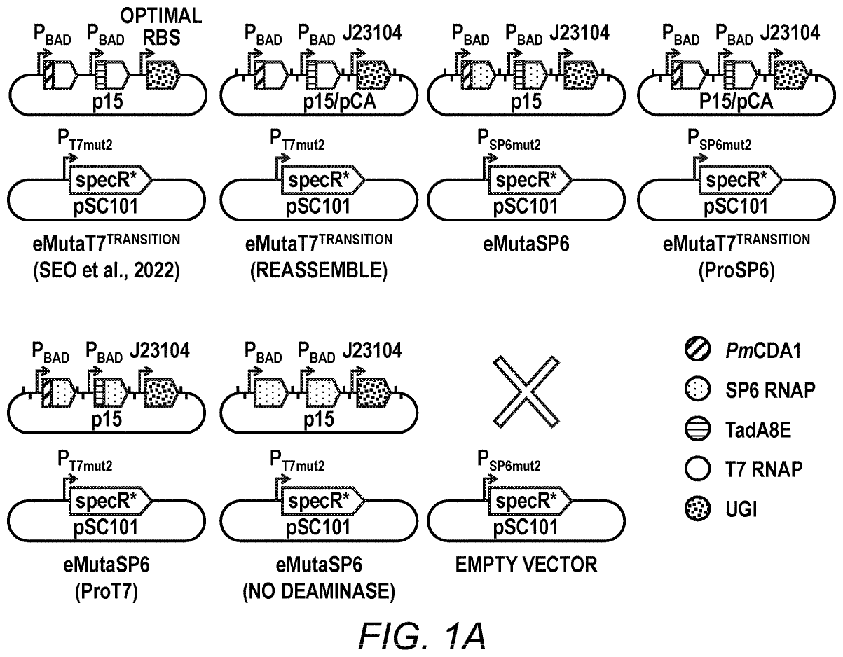

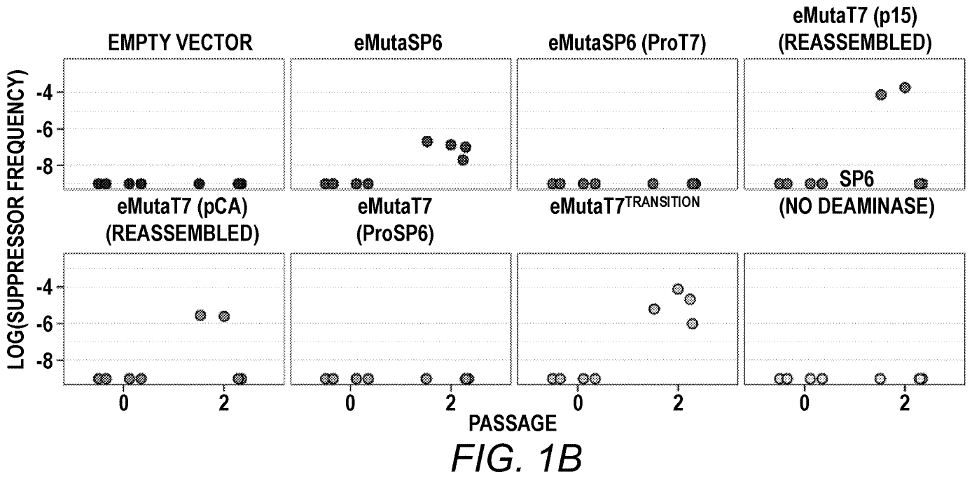

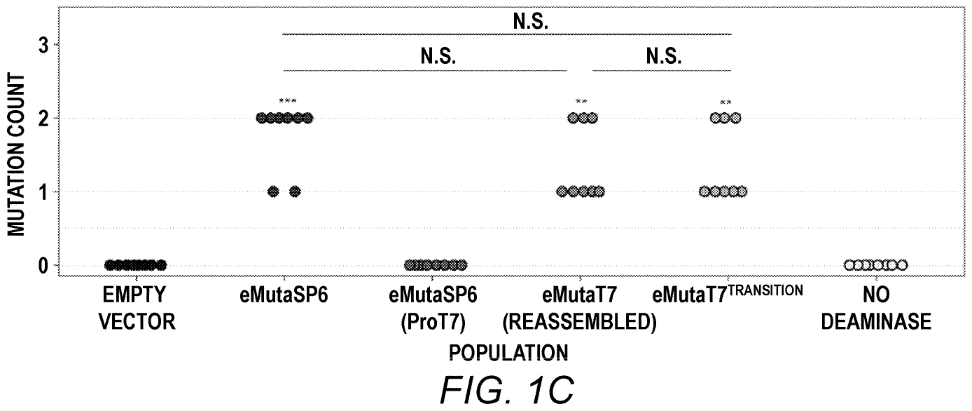

To test whether SP6 RNAP functions as a viable alternative to T7 RNAP, mutant “eMutaSP6” (FIG. 1A) was created and a specR mutant (L13*) was cloned under SP6ₘᵤₜ₂ into a low copy (SC101-nonTS) backbone, pJUMP27 (Csörgó et al. (2012) Microb. Cell Factories 11(1):11). Notably, pJUMP27 was modified to contain AmpR instead of KanR, to enable compatibility of target and mutator plasmids, which is also included in the toolkit. eMutaT7^{transition} was also reassembled using Golden Gate parts to validate the modularity of the tool. Using this approach, Level 3 constructs were made and verified within a 10-day period (FIG. 1A). The selection scheme was tested with the published eMutaT7^{transition} and it was verified that the tool was able to re-evolve the stop codon.

During vector assembly, a high frequency of aberrant plasmids were observed when using two or more spacer parts, leading to concatemers, multi-backbone or multi-marker plasmids. This was especially prevalent in Level 2 assemblies. It was hypothesized that this may be due to recombination caused by the identical sequences of the Loop kit spacer. Accordingly, new spacers were created based on ~200 bp regions of GFP and other parts which should not lead to unwanted effects. When using these, recombination events were no longer observed indicating that Golden Gate assembly efficiency may be improved by avoiding part repetition (Pollak et al. (2020) Synth. Biol. 5(1):ysaa001).

To test SP6, eMutaSP6 was constructed to be arabinose-inducible using the P_{BAD} promoter from Cyanogate kit (Vasudevan et al. (2019) Plant Physiol. 180(1):39-55), including uracil N-glycosylase inhibitor (UGI), a DNA repair inhibitor, expressed from the promoter J23104 into the p15 backbone plasmid pJUMP26 (eMutaSP6). Additionally, it was hypothesized that higher plasmid copy number would lead to an increase in mutation rate. Therefore, this same construct was cloned into the Loop backbone pCAo-1, containing the origin of replication pBR322 (Pollak et al. (2019) New Phytol. 222(1):628-40). Controls were constructed that lacked the fused deaminases and the mutator altogether, as well as ones with swapped promoters. Finally, eMutaT7^{transition} (Seo et al. (2023) Nucleic Acids Res. 51(10):e5919) was used as a positive control (FIG. 1A).

An increase in the colony number was observed in selective media, which was not observed in control constructs. This effect was also present in experiments where the stop codon could not be reversed to another codon by the deaminase, suggesting that resistance could have stemmed from genomic mutations, a common phenomenon (Valenzuela-Ortega & French (2021) Synth. Biol. Oxf Engl. 6(1):ysab003). Colony number was also increased in experiments when UGI was present.

However, it was also observed that the mutator-carrying populations had growth in selective conditions at P0, potentially due to leaky expression from pBAD, which was further supported by the presence of mutations at this timepoint. Mutations were likely to have appeared during this passage as no growth could be observed when plating directly on spectinomycin following cotransformation. Further investigation revealed that the template for the pBAD part was missing the Shine Dalgarno sequence and lacked araC. Despite this, it appeared that the promoter allowed for expression but not for repression. Accordingly, the pBAD part was modified to include araC and the Shine Dalgarno sequence. In addition, another version of the pBAD part lacking araC was created for stacking inducible constructs. To provide for tighter repression, the rhamnose-inducible promoter PrhaB (Csörgó et al. (2012) Microb. Cell Factories 11(1):11) was also included.

With the updated constructs, the evolution was repeated in E. coli BL21 cells and an improved repression pattern at passage 0 was observed. Fluctuation assay trends remained similar across constructs, although a substantial number of colonies emerging toward latter passages was observed in control constructs. Sequencing the target amplicon of randomly picked colonies from the final passage, P14 (7 days of evolution), showed that both eMutaSP6 and the reassembled eMutaT7 were able to introduce mutations into the gene of interest without any upstream or downstream mutations. However, the number of mutation was not to the level of eMutaT7^{transition} in these experiments, possibly due to the different degrees of UGI expression. However, mutations were also identified in controls, suggesting that colonies in control lines may be linked to random mutagenesis stemming in the already mutation-prone BL21 cells (Csörgó et al. (2012) Microb. Cell Factories 11 (1): 11).

The same constructs were then tested in a different bacterial system more suitable to DNA sequence preservation, namely E. coli strain 5a. Using this strain, no colonies appeared in the control lines (FIG. 1B) at timepoints previously shown to have colonies in E. coli BL21. On the other hand, colonies appeared in the construct carrying lines (FIG. 1B), but to a lesser degree than was observed in BL21. No mutations accumulated in the control lines lacking a mutator, nor did they appear in the swapped promoter line; but mutations were present in the mutator carrying lines (FIG. 1C).

Overall, these results indicate that deaminase-fused SP6 functions as an alternative to T7 in RNAP-based continuous directed evolution. It can specifically introduce mutations into a gene of interest under the SP6 promoter, allowing for T7-driven protein expression in tandem with continuous directed evolution. In addition, the toolkit enables researchers to create customizable directed evolution plasmids that contain the necessary evolution and ancillary selection parts rapidly, with uniquely needed parts easily added, increasing this technique's applicability. Furthermore, it also permits more complex schemes, such as the parallel evolution of two genes, outcome-dependent sequential evolution, or porting to other organisms.

What is claimed is:

1. A system for continuous directed evolution, comprising
(a) a first expression construct comprising a SP6 promoter operably linked to a polynucleotide encoding a first protein of interest; and
(a) a second expression construct comprising at least one polynucleotide encoding a SP6 RNA polymerase-deaminase fusion protein.

2. The system of claim 1, wherein the second expression construct comprises a polynucleotide encoding a SP6 RNA polymerase-cytosine deaminase fusion protein and a polynucleotide encoding a SP6 RNA polymerase-adenine deaminase fusion protein.

3. The system of claim 1, wherein the first expression construct and/or second expression construct further comprises a polynucleotide encoding a DNA repair inhibitor.

4. The system of claim 1, wherein the first expression construct or second expression construct further comprises a T7 promoter operably linked to a polynucleotide encoding a second protein of interest.

5. The system of claim 1, further comprising a third expression construct comprising a T7 promoter operably linked to a polynucleotide encoding a second protein of interest.

6. A host cell comprising the system of claim 1.

7. A kit comprising
(a) a polynucleotide encoding a SP6 RNA polymerase,
(b) a polynucleotide encoding a cytosine deaminase and/or, a polynucleotide encoding an adenine deaminase, and
(c) a SP6 promoter.

8. The kit of claim 7, further comprising (d) a polynucleotide encoding a DNA repair inhibitor.

9. The kit of claim 8 further comprising (e) a T7 promoter.

10. The kit of claim 7, wherein (a)-(c) are compatible with Golden Gate assembly.

11. The kit of claim 8, wherein (d) is compatible with Golden Gate assembly.

12. The kit of claim 9, wherein (e) is compatible with Golden Gate assembly.

13. A method of continuous directed evolution of a protein of interest, comprising
(a) introducing into a host cell a first expression construct comprising a SP6 promoter operably linked to a polynucleotide encoding the protein of interest;
(b) introducing into the host cell a second expression construct comprising at least one polynucleotide encoding a SP6 RNA polymerase-deaminase fusion protein; and
(c) culturing the host cell under conditions suitable for continuous directed evolution of the protein of interest.

14. The method of claim 13, wherein the second expression construct comprises a polynucleotide encoding a SP6 RNA polymerase-cytosine deaminase fusion protein and a polynucleotide encoding a SP6 RNA polymerase-adenine deaminase fusion protein.
